(19) United States
(12) Patent Application Publication
(10) Pub. No.: US 2018/0055848 A1
(43) Pub. Date: Mar. 1, 2018

(54) Combination of Albuterol and Caffeine as Synergistic Treatment for Obesity or Sarcopenia

(71) Applicant: Frank L. Greenway, Baton Rouge, LA (US)

(72) Inventor: Frank L. Greenway, Baton Rouge, LA (US)

(73) Assignee: Board of Supervisors of Louisiana State University and Agricultural and Mechanical College, Baton Rouge, LA (US)

(21) Appl. No.: 15/565,367

(22) PCT Filed: Jan. 28, 2016
(86) PCT No.: PCT/US2016/015395

§ 371 Date: Oct. 9, 2017

Related U.S. Application Data
(60) Provisional application No. 62/270,897, filed on Dec. 22, 2015.
(60) Provisional application No. 62/147,151, filed on Apr. 14, 2015.

(51) Int. Cl. A61K 31/522; A61K 31/137; A61K 9/00

(52) U.S. Cl. CPC A61K 31/522; A61K 9/0053; A61K 31/137

(57) ABSTRACT

A combination of albuterol and caffeine can be used to reduce fat body mass or to increase lean body mass. The combination may be used to treat conditions such as obesity or sarcopenia. There is a substantial synergy in the combinations: The combined effect of albuterol and caffeine is significantly greater than would be expected from the known properties of the individual components.

### PRIORITY CLAIM

The benefit of the 14 Apr. 2015 filing date of U.S. provisional patent application Ser. No. 62/147,151, and the benefit of the 22 Dec. 2015 filing date of U.S. provisional patent application Ser. No. 62/270,897 are claimed under 35 U.S.C. § 119(e) in the United States, and are claimed under applicable treaties and conventions in all countries. The complete disclosures of both priority applications are hereby incorporated by reference in their entirety.

### STATEMENT REGARDING FEDERALLY SPONSORED RESEARCH OR DEVELOPMENT

This invention was made with Government support under grants number 2P30DK072476, number P50AT002776, and number 1U54GM104940 awarded by the National Institutes of Health. The United States Government has certain rights in the invention.

### TECHNICAL FIELD

This invention pertains to a therapeutic treatment for obesity or sarcopenia in children, adolescents, and adults.

### BACKGROUND ART

### Childhood and Adolescent Obesity

The prevalence of obesity in adults requires no citation. The prevalence of childhood and adolescent obesity has risen dramatically over the past thirty years. Childhood and adolescent obesity is associated with earlier onset of type 2 diabetes, high blood pressure, and vascular disease. Childhood and adolescent obesity is not only associated with medical problems, it also often causes a decreased quality of life due to the effects of bullying and social discrimination.

Current prescription pharmacological treatments for obesity in adults include Orlistat, lorcaserin, topiramate-phentermine, liraglutide, bupropion-naltrexone, phentermine, and related compounds. Furthermore, lorcaserin, phentermine and related medications are scheduled by the DEA as having the potential for abuse, and are only approved for short-term use in adults.

Of these compounds, only Orlistat is approved by the FDA for use in adolescents. No obesity drug is currently approved for use in pre-adolescent children. Orlistat is only minimally effective for treating obesity, and it is poorly tolerated due to its gastrointestinal side effects. Food restriction in childhood and adolescence is not only difficult to accomplish, but it also raises concerns about growth and development. There is an unfilled need for a medication to treat obesity in children and adolescents by reducing body fat and increasing lean tissue, without needing to restrict food intake. A drug for treating childhood and adolescent obesity that increases lean tissue, decreases fat tissue, and does not require a change in food intake would be welcomed by pediatricians who are concerned about food restriction in growing individuals, and by children and adolescents who can be stigmatized by obesity.

Caffeine is a food ingredient found in coffee, tea, kola nuts, and other botanical sources. Caffeine is added to many soft drinks. It is approved as a non-prescription stimulant to combat drowsiness at up to 200 mg per dose or up to 1600 mg per day for people 12 years of age or older. A typical cup of coffee contains about 95-200 mg caffeine. A typical cup of brewed tea contains about 55 mg caffeine; and a 355 mL serving of some soft drinks contains about 45-55 mg caffeine.

Caffeine is known to stimulate lipolysis and metabolic rate. Caffeine inhibits phosphodiesterase. 100 mg of caffeine increases the resting metabolic rate by ˜3-4% (150 kcal/day in lean adults and 80 kcal/day in post-obese adults). It increases intracellular cyclic adenosine monophosphate (cAMP) levels by competitively inhibiting phosphodiesterase, an enzyme that breaks down cAMP. Increased cAMP results in increased triacylglyceride breakdown, and increased circulating free fatty acids (FFA), thus regulating lipolysis in white adipose tissue (WAT). Caffeine also stimulates the sympathetic nervous system, and activates the 32 adrenergic receptors on WAT cells through neural release of norepinephrine. The activation of the 132 adrenergic receptors results in the activation of adenylate cyclase, promotion of cAMP production, and lipolysis. Even though caffeine increases resting energy expenditure and increases fatty acid turnover, most of the mobilized FFAs are eventually re-esterified, meaning that caffeine alone produces no significant weight loss, fat loss, or gain in lean tissue. There is an unfilled need for a safe medication to complement the effects of caffeine, to achieve weight loss, fat loss, and gain in lean tissue.

The structure of caffeine is:

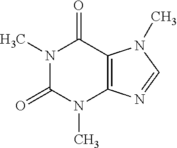

Ephedrine, an alpha- and beta-adrenergic receptor agonist, was originally marketed as a bronchodilator. Ephedrine releases norepinephrine from nerve terminals, and stimulates both alpha- and beta-adrenergic receptors. Ephedrine was later found to increase thermogenesis and metabolism in asthmatic patients, and it has been investigated as a potential weight loss treatment. Ephedrine has been combined with caffeine to complement caffeine's effect on lipolysis. However, in 2004 the FDA banned the sale of all ephedrine-containing supplements due to concerns over cardiovascular risks. The combination of caffeine and ephedrine also has the disadvantage of increasing blood pressure. The sale of ephedrine is now heavily regulated due to its potential use in manufacturing the illicit drug methamphetamine.

Albuterol (salbutamol) is a selective beta-2 adrenergic agonist used to treat asthma. Albuterol may be a safer alternative to ephedrine, because albuterol does not stimulate alpha-adrenergic-associated vasoconstriction, and albuterol therefore does not increase blood pressure. When given by inhalation at four times the therapeutic dose of 200 μg, albuterol increases metabolic rate, increases oxygen consumption, and stimulates lipolysis. See P. Amoroso et al., “Acute effects of inhaled salbutamol on the metabolic rate of normal subjects,” Thorax, 1993 September; 48(9):882-5; and R. Goldberg et al., “Metabolic responses to selective beta- adrenergic stimulation in man,” Postgraduate Medical Journal, 1975 February; 51(592):53-8. However, such a high dose given via aerosol almost invariably causes tachycardia. Oral administration of albuterol does not cause this side effect, and thus an oral pill seems to be safer. Albuterol has been approved for ages 2 and older for treating asthma. Albuterol has also been shown to increase muscle strength and lean body mass in children with spinal muscular atrophy, and in healthy young men during an exercise training program. The suggested dose for treating asthma in adults and adolescents (over age 12) is 4 mg albuterol three or four times a day, and for children between 6 and 12 years the suggested dose is 2 mg albuterol three or four times a day. For children between the ages of 2 and 5, the suggested dose is 0.1 mg/kg body weight three times a day, with a maximum dose of 2 mg three times a day.

The structure of albuterol (salbutamol) is:

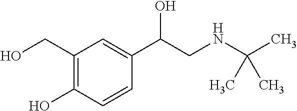 [SMILES: CC(C)(C)NCC(O)c1ccc(O)c(CO)c1]

Salbutamol has two optical isomers. It is typically synthesized and sold as a racemic mixture. The (R)-enantiomer is believed to be responsible for the pharmacologic activity. The (S)-enantiomer is believed to block metabolic pathways that are associated with elimination of both enantiomers.

### Sarcopenia

Sarcopenia is the loss of muscle tissue as one ages. Humans typically lose about 1-2% of muscle mass annually after age 50, a rate that increases to about 4% annually after age 60. Body weight does not change dramatically, however, because the percentage of body fat increases. This loss of muscle tissue can lead to frailty, falls and other injuries, and loss of ability to live independently. Associated annual costs to the United States health care system were reported to be about $18 billion as of 2004, and costs in the rest of the world, although not figures are not readily available, are presumably much higher. There is currently no approved medical treatment for sarcopenia or for frailty. There is an unfilled need for a medication to treat sarcopenia by increasing lean tissue, preferably without also increasing body fat, and without needing to restrict food intake. A treatment for sarcopenia would help to increase muscle mass and reduce frailty in the elderly, and to reduce falls that can result in life-threatening hip fractures, other injuries, and to treat muscle-wasting diseases such as muscular dystrophy.

Anabolic steroids have been used to increase muscle mass, but have also been associated with liver problems and with lipid changes associated with increased risk of cardiovascular disease. The use of anabolic steroids has been restricted due to their dangers combined with their potential for abuse.

R. Butcher et al., “Effects of lipolytic and antilipolytic substances on adenosine 3′,5′-monophosphate levels in isolated fat cells,” J. Biol. Chem., vol. 243, pp. 1705-1712 (1968) reported that combined treatment with catecholamines and caffeine produced a synergistic increase in cAMP production in adipocytes.

J. Wenkeova et al., “Adrenergic lipolysis in human adipose tissue in vitro,” Eur. J. Pharmacol., vol. 30, pp. 49-55 (1975) reported that a2-adrenergic receptors inhibited WAT lipolysis.

F. Borsine et al. Life Sci. 1982;30(11):905-11 reported that salbutamol (i.e., albuterol) induced a dose-related decrease in food intake in rodents. (Note that these findings contradict certain findings reported here.)

J. Choo et al. Am J Physiol. 1992;263(Pt 1):E50-6 reported that albuterol increased lean tissue in rodents when albuterol was administered intravenously or subcutaneously.

J. Caruso et al., Oral albuterol dosing during the latter stages of a resistance exercise program. Journal of strength and conditioning research/National Strength & Conditioning Association. 2005 February; 19(1):102-7 reported that administering albuterol during the latter stages of a resistance exercise program resulted in significantly higher strength gains in certain muscles versus placebo; and that there was a trend toward higher lean body mass.

C. Skura et al., Albuterol increases lean body mass in ambulatory boys with Duchenne or Becker muscular dystrophy. Neurology. 2008 Jan. 8; 70(2):137-43 reported that short-term treatment with an extended release formulation of albuterol may increase lean body mass, decrease fat mass, and improve functional measures in patients with dystrophinopathies such as Duchenne or Becker muscular dystrophy.

E. Uc et al., Albuterol improves response to levodopa and increases skeletal muscle mass in patient with fluctuating Parkinson disease. Clin Neuropharmacol. 2003;26(4):207-12 reported that albuterol increased muscle mass and improved therapeutic response to levodopa in patients with fluctuating Parkinson disease. A clear anabolic effect was seen, as evidenced by a significant increase in thigh muscle mass and whole-body fat-free mass. There was no increase in muscle strength, despite the increased muscle mass. The authors said that a double-blind, placebo-controlled study was needed to confirm the effects.

### SUMMARY OF THE INVENTION

I have discovered that therapy with a combination of both albuterol and caffeine can effectively treat obesity. I have discovered that therapy with a combination of both albuterol and caffeine can effectively treat sarcopenia. There is a substantial synergy: The combined effect of albuterol and caffeine is significantly greater than would be expected from the known properties of the two individual components. Albuterol and caffeine are inexpensive and well tolerated, even in subjects with congestive heart failure.

In one embodiment, the two compounds are admixed and administered as a combination pill or capsule containing an albuterol : caffeine ratio of 1:25, for example 4 mg of albuterol and 100 mg of caffeine, which can be given orally 3 times a day. Initial trials (for both obesity and sarcopenia) will be conducted with an immediate release formulation of the compounds.

In another embodiment a timed-release formulation pill containing an albuterol:caffeine ratio of 1:25, for example 12 mg of albuterol and 300 mg of caffeine, is taken once a day, typically in the morning, lasting throughout the day, and waning at bedtime to minimize insomnia. Methods of making timed-release formulations are well known in the art, and any of these known methods may be used in practicing this aspect of the invention.

In one embodiment, the formulation is used to treat obesity.

In another embodiment, the formulation is used to treat sarcopenia.

Albuterol can potentially cause insomnia, anxiety and tremors, but it should not cause the hypertension or tachycardia that have previously been seen with ephedrine. Albuterol should not alter appetite, but it should increase lipolysis and it should increase lean tissue. The increase in lean tissue appears to be an acute effect, resulting from the down-regulation of certain receptors. The combination of caffeine and albuterol should have a minimal effect on food intake. The combination is synergistic in increasing metabolic rate, increasing lipolysis, and increasing lean tissue. Without wishing to be bound by this hypothesis, I propose that caffeine inhibits the down-regulation of beta-2 receptors in response to albuterol, that caffeine increases cAMP levels by inhibiting phosphodiesterase, and that caffeine thus magnifies the effects of albuterol.

The invention may be used both in humans and in non-human mammals. There is an unfilled need for improved methods for treating veterinary obesity, particularly in dogs and cats. Cats and dogs tend to be more sensitive than humans to adrenergic stimulation in general, whether from caffeine or another adrenergic stimulator. Without wishing to be bound by this hypothesis, it is likely that cats and dogs will respond to the same ratio of albuterol and caffeine to which humans and rodents respond (between 1:20 and 1:30, preferably 1:25), but that the dose would need to be reduced more than simply in proportion to the metabolic mass equation. Routine experimentation may be used to find the optimal dosage for beneficial effects in cats and dogs without toxicity.

### BRIEF DESCRIPTION OF THE DRAWINGS

FIG. 1 depicts observed lipolysis levels following different treatments for adipocytes in vitro.

FIG. 2 depicts the effects of various doses of caffeine, albuterol, or a combination on human metabolic rates.

FIGS. 3A-F depict the effects of caffeine and albuterol on body composition and metabolic rate in rats. FIG. 3A depicts weight gain. FIG. 3B depicts food intake. FIG. 3C depicts total activity. FIG. 3D depicts body composition. FIG. 3E depicts energy expenditure. FIG. 3F depicts respiratory exchange ratios.

FIGS. 4A-D depict changes in lean and fat mass in rats with caffeine/albuterol compared to albuterol alone. FIG. 4A depicts lean mass gain. FIG. 4B depicts change in lean mass percentage. FIG. 4C depicts fat mass gain. FIG. 4D depicts change in percent fat mass.

### MODES FOR CARRYING OUT THE INVENTION

In preliminary tests in rodents, a combination that was the human equivalent (adjusting for body mass) to 4 mg albuterol and 100 mg caffeine, given three times a day, increased fat loss and increased lean tissue gain significantly more than either caffeine or albuterol alone, and significantly more than would be expected from the combined effects of the individual components, demonstrating the synergy of the combination.

The preferred mass ratio of albuterol to caffeine is 1:25, with a range between 1:20 and 1:30. The preferred dose will vary depending on the age and body mass of the patient; for most adolescents and adults the preferred amount of caffeine is 100 mg, administered three times daily, along with a proportionate amount of albuterol, preferably 4 mg. Doses will be proportionately smaller in pre-adolescent children.

The combination of caffeine with albuterol should not affect blood pressure. The novel combination increases metabolic rate in a safe manner. Unlike earlier combinations involving caffeine and ephedrine, the novel caffeine/albuterol combination does not have any known potential for use in manufacturing illegal drugs.

In a series of experiments using cultured adipocytes, rat models, and humans, the effects of caffeine and albuterol on lipolysis, metabolic rate, food intake, and body composition have been evaluated. Both caffeine and albuterol enhanced lipolysis in cultured adipocytes. Acute treatment of humans with caffeine, albuterol, or a combination increased the resting metabolic rate. Longer-term studies of rats revealed a trend for increased metabolic rate with albuterol treatment. There were increased lean mass gain and decreased fat mass gain with the combined caffeine/albuterol treatment, effects that were greater than with albuterol alone. In rats, albuterol with caffeine produced significantly greater synergistic increases in lean body mass and reductions in fat mass without changes in food intake after 8 weeks of treatment.

### EXAMPLE 1

### Statistical Analyses

In all instances where statistical analyses are reported, data were analyzed using the PROC MIXED procedure of the SAS® version 9.3 statistical software package (SAS Institute, Inc., Cary, N.C.). Data from Example 2 were analyzed by analysis of variance. Example 4 investigated 8 treatments (various combinations of albuterol and caffeine) in an 8×8 Latin square study design, with 8 subjects observed across 8 weeks in a balanced arrangement of the 8 treatment-dose combinations, in which each subject received each of the treatment-dose combinations exactly once, and each treatment-dose combination was given exactly once each week. In these pilot trials, no attempts were made to determine the number of participants required to provide a nominal 80% power for detecting specific treatment differences. In Example 4, changes from assessment time 0 were viewed as repeated measurements across assessment times (60, 120, 180 minutes), and modeled as effects of albuterol dose, caffeine dose, treatment week, and assessment time. Demographics, adverse events and any other non-normally distributed data were analyzed by a chi-squared test. Data from Examples 5 and 6 were analyzed similarly, using analogous mixed-effects statistical models. Energy expenditure data were analyzed using analysis of covariance, with lean body mass as a covariate. Statistical significance was defined as p 5 0.05. Data in other Examples are analyzed similarly.

### EXAMPLE 2

### Lipolysis Assay

Human fat cells in culture increased lipolysis in the presence of caffeine, and also increased lipolysis in the presence of albuterol. The largest increase in lipolysis was seen with a combination of albuterol and caffeine in a 1:25 mass ratio, although in this early experiment the increase was not significantly different from adding the expected effects of the corresponding doses of albuterol and caffeine.

Primary human adipose tissue was obtained from patients undergoing liposuction, paniculectomy, or bariatric surgery. Tissue was processed by LaCell LLC (New Orleans, LA) to isolate and cryopreserve pre-adipocytes. At the time of the assay, pre-adipocytes were differentiated into adipocytes. Differentiated human adipocytes adherent to the bottom of a 96-well plate were washed and treated with media containing one of ten treatments: (1) albuterol 7 ng/mL, (2) albuterol 17 ng/mL, (3) caffeine 3 μg/mL, (4) caffeine 10 μg/mL, (5) albuterol 7 ng/mL and caffeine 3 μg/mL, (6) albuterol 17 ng/mL and caffeine 3 μg/mL, (7) albuterol 7 ng/mL and caffeine 10 μg/mL, (8) albuterol 17 ng/mL and caffeine 10 μg/mL, (9) Isoproterenol 1 μM as a positive control, or (10) buffer only as a negative control. Cells were incubated with the respective treatments at 37° C., under a 5% CO₂ atmosphere for 3 hours. After incubation, a 50 μL aliquot was removed and combined with 50 μL glycerol reagent, in a colorimetric assay based on the amount of glycerol released during incubation with test compounds. Absorbance was measured at 540 nm, and results were calibrated against a standard curve. Eight replicates were conducted for each treatment.

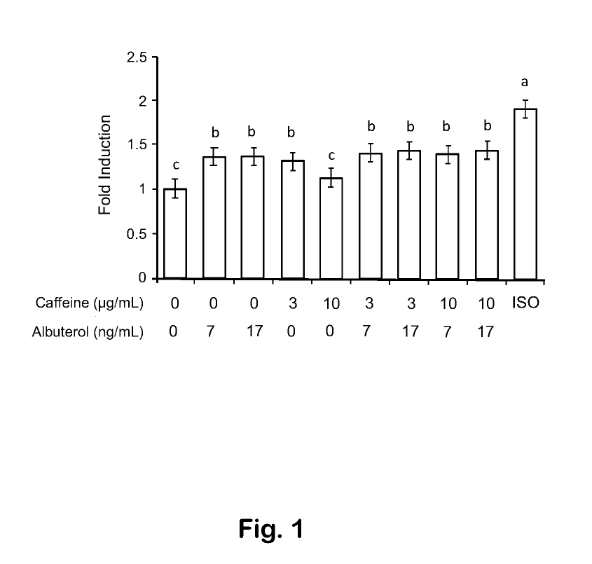

Treatment of adipocytes with any dose of caffeine, albuterol, or combination resulted in ˜30-40% increase in lipolysis over buffer-treated cells (p<0.01), with the curious exception of the 10 μg/mL of caffeine treatment (p=0.83). These increases were smaller than the ˜90% increase in lipolysis seen with isoproterenol treatment (p<0.001). FIG. 1 depicts observed lipolysis for the different treatments. Values in FIG. 1 are mean±95% confidence interval, n=8. Measurements that were significantly different (p<0.05) are denoted by different letters. Measurements with the same letter were not significantly different.

### EXAMPLE 3

### Pilot Test in a Single Volunteer

An adult male (66 years old) who took 4 mg albuterol plus caffeine 100 mg orally three times daily (tid) for two months increased lean mass by 1.25% and decreased fat mass by 1.2%. These effects are expected to be even greater in a growing child or adolescent.

### EXAMPLE 4

### Metabolic Rate in Humans

A preliminary trial in 8 humans showed that a dose of 100 mg caffeine combined with 4 mg albuterol increased metabolic rate to a degree that was numerically greater than would be expected from adding the expected effects of the individual components; however, with this small sample size the increase was not statistically significant. A combination of 100 mg caffeine with 4 mg albuterol appeared to be optimal, within the scope of this preliminary trial.

The study was approved by the Pennington Biomedical Research Center Institutional Review Board. Eight healthy adults (both males and females) between the ages of 18 and 50 years, with body mass indices between 19 and 40 kg/m², were recruited from the greater Baton Rouge, La. area. Specifically excluded were any subjects who were pregnant, breast feeding, smoking, using nicotine products, taking regular medications, or taking any medication known to alter metabolic rate such as an asthma medication or a beta-adrenergic blocking drug. Also excluded were women of child-bearing potential who did not agree to use an effective method of contraception (abstinence, barrier methods, intrauterine devices, or hormonal methods of contraception). All subjects gave written informed consent prior to beginning the study.

The study was a randomized, double-blind, crossover study comparing the effects of: (1) albuterol 2 mg—placebo, (2) albuterol 4 mg—placebo, (3) placebo—caffeine 100 mg, (4) placebo—caffeine 200 mg, (5) albuterol 2 mg—caffeine 100 mg, (6) albuterol 2 mg—caffeine 200 mg, (7) albuterol 4 mg—caffeine 100 mg, and (8) albuterol 4 mg—caffeine 200 mg. Each subject completed a total of eight test days; consecutive test days were separated from one another by approximately one week (viz., 7±2 days). Before each visit (between 7 a.m. and 9 a.m., typically 8 a.m.), the subjects fasted from 9 p.m. the previous evening, refrained from strenuous physical activity for 24 hours, otherwise ate their usual diets, and refrained from alcohol and caffeine-containing beverages for 48 hours. On each visit, blood pressure, pulse rate, and temperature were recorded; and each subject rested for 30 minutes prior to measurement of the resting metabolic rate (RMR) and respiratory quotient (RQ) at 0, 60, 120 and 180 minutes. Resting metabolic rate and respiratory quotient were measured by indirect calorimetry using a ventilated hood system (DeltaTrac II metabolic monitor, Datex Inc. Helsinki, Finland). A transparent hood was placed over the subject's head and the amount of oxygen consumed and carbon dioxide exhaled were measured. After baseline measurements had been taken, the subjects were given the designated treatment, containing 2 pills to swallow, and baseline measurements were then repeated for the last 30 minutes of each hour for the next 3 hours.

Eight subjects completed this eight-condition cross-over study. Characteristics of the subjects are summarized in Table 1.

TABLE 1
Characteristics of Study Subjects Participating
in the Tests of Example 4

| Gender | 7 female, 1 male |
|---|---|
| Ethnicity | 5 Caucasian, 3 African American |
| Age (years) | 32 ± 7 |
| Weight (kg) | 71.8 ± 11.8 |
| Body mass index (kg/m²) | 25.0 ± 4.9 |
| Systolic blood pressure (mm Hg) | 110 ± 7 |
| Diastolic blood pressure (mm Hg) | 74 ± 8 |
| Pulse (beats per minute) | 67 ± 12 |

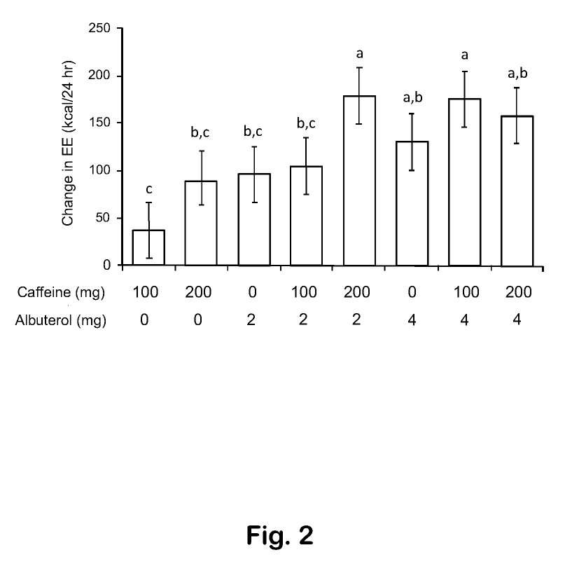

All dosages of caffeine, albuterol, or a combination resulted in significant increases in energy expenditure over the baseline metabolic rate of 1368±153 kcal/d (p<0.02 to p<0.001, see FIG. 2). FIG. 2 depicts the effects of various doses of caffeine, albuterol, or a combination on human metabolic rates. Values shown in FIG. 2 are mean±95% confidence interval, n=8. Measurements that were significantly different (p<0.05) are denoted by different letters (a, b, c). Measurements with the same letter were not significantly different from one another.

Two of the caffeine/albuterol combinations increased energy expenditure more than twice the increase seen with the corresponding caffeine monotherapy. The increased energy expenditure (the “delta”) was 2.9 times greater with a combination therapy of 2 mg albuterol and 100 mg caffeine as compared to 100 mg caffeine alone (105 kcal versus 37 kcal). Increased energy expenditure was 4.8 times greater with a combination therapy of 4 mg albuterol and 100 mg caffeine compared to 100 mg caffeine alone (175 kcal versus 37, p<0.0001). Although there was evidence that the combination therapy was significantly better than the monotherapy, dose-response relationships were inconclusive in this small-scale test.

No statistically significant changes were seen in systolic blood pressure, diastolic blood pressure, heart rate, or temperature (data not shown).

### EXAMPLES 5 AND 6

### Weight Gain and Body Composition in Rats

The Pennington Biomedical Research Center Animal Care and Use Committee approved the animal protocols. Rats were individually housed in shoe-box cages under controlled conditions (12 h light-dark cycle, 22° C., 55% humidity). Male Sprague-Dawley rats (Harlan, Inc., Indianapolis, Ind., 8-9 weeks old) were fed a high fat diet (60% calories from fat, D12492, Research Diets, New Brunswick, N.J.) for 4 weeks. For Example 5, forty rats were randomized to 4 treatment groups and continued on the high-fat diet for 4 weeks. The treatments for Example 5 were: (1) saline/saline, (2) saline/albuterol, (3) saline/caffeine, and (4) caffeine/albuterol. For Example 6, sixty rats were randomized to 2 treatment groups and continued on the high-fat diet for 8 weeks. The treatments for Example 6 were: (1) saline/albuterol and (2) caffeine/albuterol. All treatments were administered by intraperitoneal injection twice a day. Albuterol was administered at 0.125 mg/kg, and caffeine was administered at 3.12 mg/kg. These doses were the rodent equivalent of administering to a human albuterol 4 mg and caffeine 100 mg three times a day, based on the Kleiber metabolic mass equation. (See Keesey R E and Corbett S W. Int J Obes. 1990;14(12):1079-84). Body composition was measured by Nuclear Magnetic Resonance (Minispec LF90 NMR analyzer, Bruker Optics, Billerica, Mass.). Weight and food intake were recorded every 2 days. In Example 5, the animals were placed in a metabolic chamber (Phenomaster, TSE Systems, Chesterfield, Mo.) at the end of the 4 week treatment period for 3 days to measure oxygen consumption, respiratory exchange ratio, and activity levels, during which time the treatment to which they had been assigned was continued. Total activity was measured with a Phenomaster system (TSE Systems, Chesterfield Mo.). Chambers had evenly spaced infrared beam grids along X, Y, and Z axes; the system sensed and quantified total beam breaks caused by movements of the animal.

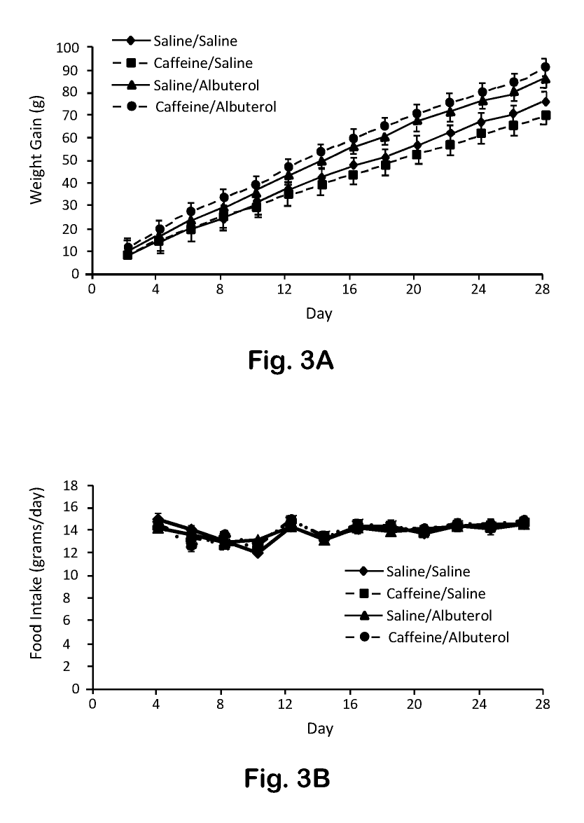

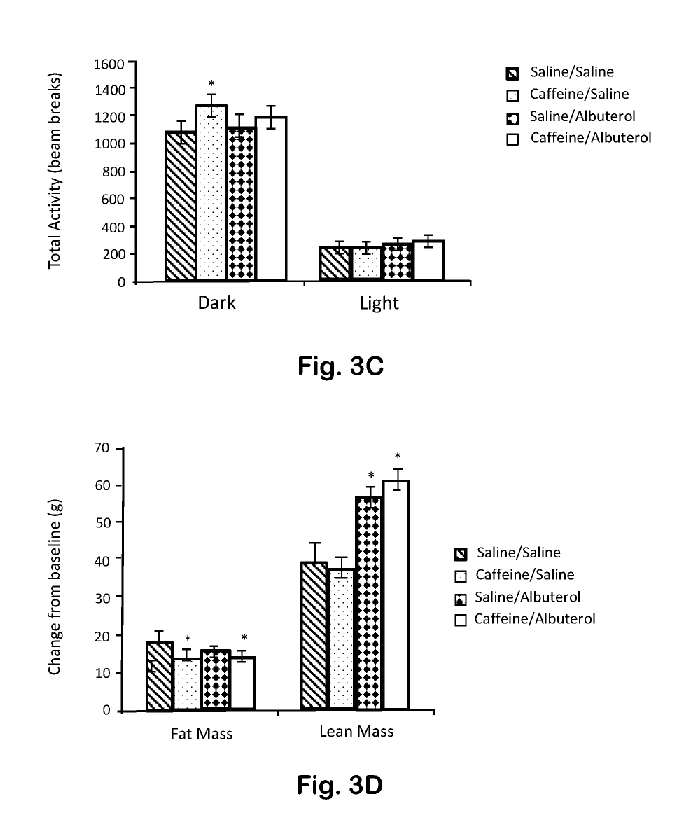

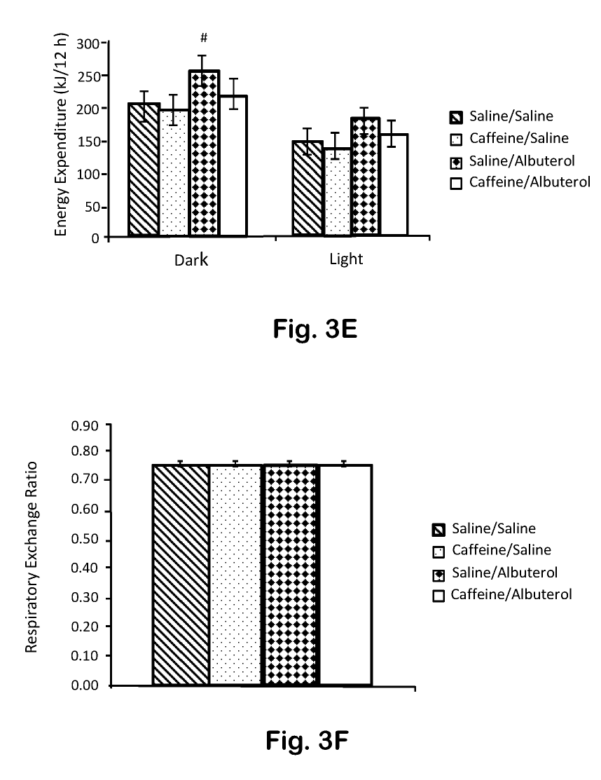

FIGS. 3A-F depict the effect of caffeine and albuterol on body composition and metabolic rate in rats. FIG. 3A depicts weight gain. FIG. 3B depicts food intake. FIG. 3C depicts total activity. FIG. 3D depicts body composition. FIG. 3E depicts energy expenditure. FIG. 3F depicts respiratory exchange ratios. Values shown in FIGS. 3A-F are mean±95% confidence interval, n=8-10. *p<0.05. # p=0.07.

Rats treated with saline/albuterol or caffeine/albuterol gained significantly more weight as compared to the saline control group (p=0.04, FIG. 3A). This observation was not explained by changes in food intake or activity, as only the caffeine/saline group experienced a significant increase in activity (p=0.02, FIGS. 3B and 3C). The differences in weight were largely explained by changes in body composition. Rats treated with saline/albuterol or caffeine/albuterol gained an additional 16.6 and 20.2 g of lean mass, respectively, compared to the saline control group (p<0.001, FIG. 3D). Treatment with caffeine or caffeine/albuterol also reduced fat accretion (p=0.006, FIG. 3D). Rats treated with albuterol alone exhibited a trend towards increased energy expenditure during the dark phase (p=0.07, FIG. 3E). Respiratory exchange ratio was similar among all groups (FIG. 3F).

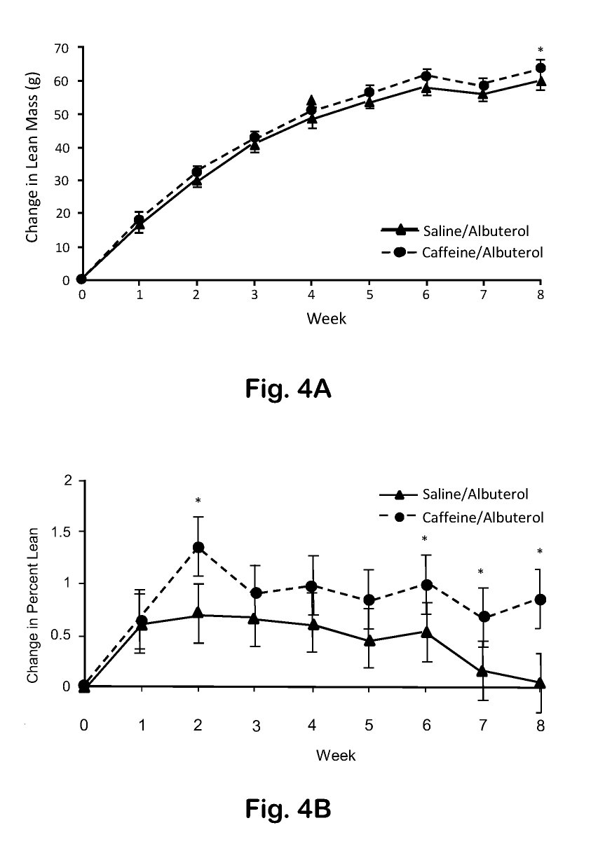

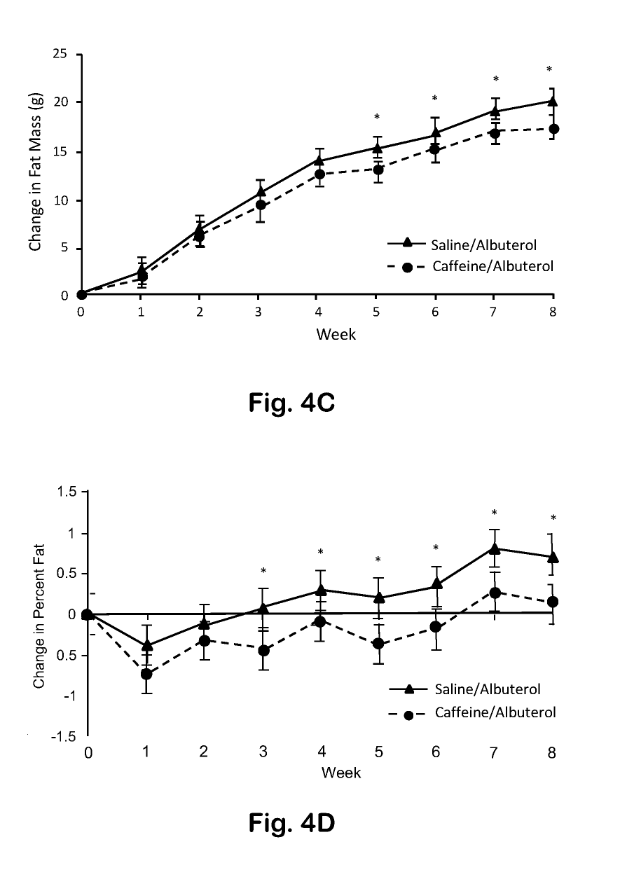

FIGS. 4A-D depict changes in lean and fat mass with caffeine/albuterol compared to albuterol alone. FIG. 4A depicts lean mass gain. FIG. 4B depicts change in lean mass percentage. FIG. 4C depicts fat mass gain. FIG. 4D depicts change in percent fat mass. Values shown in FIGS. 4A-D are mean±95% confidence interval, n=30. *p<0.05 to p<0.004.

Because rats treated with caffeine/albuterol appeared to gain more lean mass than rats treated with albuterol alone in Experiment 3, an eight-week study was undertaken to assess the effect of a longer-term treatment. Rats treated with caffeine/albuterol gained more lean mass by week 8 as compared to those treated with albuterol alone (63.9 g v. 60.3 g, p=0.03, FIG. 4A). When percentages of lean mass were compared, significant differences were detected at weeks 2, 6, 7, and 8 (FIG. 4B). Rats treated with caffeine/albuterol gained less fat mass as compared to those treated with albuterol alone (FIG. 4C). This difference was statistically significant by week 5 and persisted through week 8 (17.4 g v. 20.1 g, p=0.004). When percentages of fat mass were compared, significant differences could be detected beginning at week 3 (FIG. 4D).

### DISCUSSION, EXAMPLES 1-6

As compared to albuterol alone, the combination of albuterol and caffeine displayed synergy, producing a larger increase in cAMP, and a larger increase in glycerol production (an end product of lipolysis). Example 2 measured glycerol production of adipocytes in vitro, following treatment with different doses of caffeine, albuterol, and a caffeine/albuterol combination (FIG. 1). Other than the anomalous 10 μg/ml dose of caffeine, all treatments resulted in a ˜30% increase in glycerol production. Surprisingly, treatment with the caffeine/albuterol combinations did not result in any additive or synergistic effects on glycerol production compared to caffeine alone. This result was seemingly inconsistent with results reported by Butcher et al. (1968), who reported that cultured adipocytes treated with a combination of caffeine and the α- and β-agonist epinephrine increased production of cAMP. Because the lipolysis mechanism relies on cAMP, greater effects on glycerol production were expected with the more selective β-agonist albuterol combined with caffeine than with albuterol alone, but that is not what was observed.

The data showed that the resting metabolic rate for all treatments increased from baseline. However, the data did not show a statistically significant synergy between albuterol and caffeine in increasing metabolic rate. Response trends were inconsistent across combined doses of albuterol and caffeine.

Albuterol is a selective β2 adrenergic receptor agonist. Because α-adrenergic stimulators such as ephedrine cause vasoconstriction, inducing an acute increase in pulse rate and blood pressure, it is possible that the previously-observed synergy between ephedrine and caffeine on energy expenditure is due to stimulation of α adrenergic receptors. However, albuterol does not stimulate α adrenergic receptors. Thus it is not possible to extrapolate from observations with ephedrine to results with albuterol. The two compounds have different modes and mechanisms of action.

Example 5 showed some interesting and surprising changes in body composition in rats. The saline/albuterol and caffeine/albuterol treatment groups actually gained more weight than did the saline control group. However, the caffeine/saline and caffeine/albuterol groups showed reduced gains in body fat. These data indicated that the differences in body weight were largely due to an increase in lean body tissue with albuterol treatment. The combination of caffeine and albuterol did not increase lean body tissue significantly more than did albuterol alone in Example 5, but the difference was significant in Example 6 with a larger group size and longer treatment duration (8 weeks v. 4 weeks). These data supported the muscle-building effect of albuterol as a monotherapy, which has been previously reported. The increased skeletal muscle mass is believed to result both from increased muscle protein synthesis, and from decreased protein degradation.

The combination of caffeine and albuterol for treatment of obesity can be used for all ages; it will be particularly useful for treatment of pediatric obesity and adolescent obesity. Currently, Orlistat is the only medication approved for obesity treatment in adolescents, and it has embarrassing side effects such as fecal urgency and oily stools. Albuterol has previously been approved for the treatment of asthma in children age 2 and older. Caffeine is not only considered to be a “food,” but it is also an approved non-prescription medication for the treatment of drowsiness in children age 12 and older. Our data indicate that the combination of caffeine and albuterol can synergistically increase lean mass and decrease fat mass during growth and weight gain. A combination therapy that increases lean mass, decreases fat mass, and does so without changing food intake will be a welcome option for physicians confronted with the challenge of treating childhood or adolescent obesity. Parents are often reluctant to act as “food police,” and many pediatricians are concerned about restricting food intake during a child's growth. The novel treatment overcomes these obstacles.

### EXAMPLE 7

### Study in Adolescents

A randomized, double-blind, placebo-controlled study is conducted in 20 obese adolescents (BMI≧95th percentile, age 12-17 years). Consent and assent are discussed and obtained at the initial screening visit, and qualification for the study is determined. If the adolescent meets all of the inclusion and none of the exclusion criteria, he or she will be enrolled in the study. Body composition testing will occur before and then 8 weeks after taking the assigned intervention.

The safety of each medication, the placebo, and the combination of the two medications will be assessed. The hypothesis is that the combination of caffeine and albuterol will cause the greatest increase in lean body mass and decrease in fat mass, more than the individual medications alone, and more than the sum of the effects from the individual medications (synergy). Data from this pilot study will serve as preliminary data for a larger future study.

Participants will attend an initial screening visit (SV) after an overnight fast of 10 hours. Consent and assent will be obtained (including parental assent) and the inclusion/exclusion criteria will be assessed. Screening labs (CBC; Chem 26 including glucose, BUN, creatinine, sodium, chloride, potassium, carbon dioxide, uric acid, total protein, phosphorus, albumin, calcium, magnesium, total bilirubin, CPK, LDH, AST, ALT, alkaline phosphatase, GGT, amylase, iron, cholesterol, triglycerides, HDL, LDL; fasting insulin; and urinalysis with urine pregnancy test as needed); and an EKG will help determine eligibility.

Inclusion Criteria:
Healthy male or female with a BMI 95th percentile
Between 12 and 17 years of age inclusive
Tanner Stage III and above

Exclusion Criteria:
Pregnant, planning to become pregnant, or nursing
Taking a medication known to affect weight or body composition such as systemic glucocorticoids, atypical anti-psychotics, or weight loss medications
Taking beta-stimulators or beta-blockers on a regular basis
Taking stimulants for attention deficit disorder
Taking any chronic medication that has not had a stable dose for 1 month or longer
Type 1 or type 2 diabetic
Any significant cardiac disease (e.g., heart failure, arrhythmias, or valve disease), uncontrolled pulmonary disease, chronic liver disease, chronic kidney disease, or chronic infectious disease
Any significant psychiatric illness that is unstable or untreated, e.g., bipolar disorder, severe depression, or severe anxiety
Otherwise considered unfit to participate in the study, based on evaluation by the medical investigator

Study Design

Twenty obese adolescents will be recruited through general advertisements and physician referrals. The study will comprise six total study visits. The first study visit will be a screening visit, and will include a medical history/physical examination, an electrocardiogram, and screening labs. The Beck Depression Inventory will be used to screen for possible depression. A psychologist is available on site to assess anyone whose score on the Beck Depression Inventory suggests further evaluation, and where appropriate the adolescent will be referred to outside sources for psychological or psychiatric therapy. Adolescents who qualify will return for a baseline (fasting) visit within one month of the initial screening visit. The adolescent will have a DXA scan for body composition, as well as a Visual Analog Scale (VAS) test for hunger and satiety. A parent will complete a Vanderbilt Assessment Scale for ADHD to determine if the adolescent shows signs of attention deficit or hyperactivity at baseline. The adolescent will then be randomized to one of the four, equally-sized groups:

Group 1: Placebo-placebo three times per day (tid) orally. Group 2: Caffeine 100 mg-placebo tid orally. Group 3: Albuterol 4 mg-placebo tid orally. Group 4: Caffeine 100 mg tid and Albuterol 4 mg tid orally.

Subjects will be advised to limit their intact of any additional caffeine. It should not be necessary to restrict subjects' intake of additional caffeine for safety concerns. The 300 mg/day used in this combination is substantially less than the amount of non-prescription caffeine one could consume in accordance with package directions (up to 1600 mg/day). Nevertheless, it would be desirable to restrict other sources of caffeine to help maintain the optimal ratio of albuterol to caffeine.

Starting with the baseline visit and repeating at each 2 week visit, the adolescent and a parent will meet with a registered dietitian certified in pediatric weight management. The information to be provided by the dietitian will include recommendations on healthy eating, increasing physical activity, and family participation as recommended by the Academy of Nutrition and Dietetics. At each of weeks 2, 4, 6, and 8, compliance (via pill counts) and adverse events will be assessed. In addition to these assessments, at each of weeks 4 and 8 the adolescent will arrive at the clinic fasting, and will have a repeat VAS scale for hunger and satiety, and a parent will fill out another Vanderbilt Assessment Scale for ADHD as a safety measure. If any concerns appear based on the Vanderbilt Assessment Scale, the intervention will be stopped and the adolescent will be referred to an appropriate health care provider for further evaluation. Finally, at the last study visit, after 8 weeks of intervention, the adolescent will have an electrocardiogram, a repeat DXA, and repeat labs to compare with the initial screening labs.

Because this will be a pilot study, no power calculation will be done to determine the expected number of participants for statistical significance. Even if there should be no statistically significant difference between groups, the pilot study is nevertheless expected to show proof of concept, and to suggest the variance and differences between the groups needed to power a statistically significant follow-up study. The primary endpoint will be change in lean body mass and change in fat mass over the 8-week period via DXA scan using Student t-tests. In addition, secondary endpoints will include QTc interval changes from baseline in the three treatment groups compared with placebo using Student t-tests. Any adverse events and side effects will be compared using a chi-squared test or other appropriate statistical test. Statistical significance will be set at p<0.05; although statistical significance is not expected in this small-scale pilot study. Demographics will be compared by t-test if normally distributed. Any non-normally distributed data will be analyzed by chi-squared test.

Outcomes

Because adolescents are still growing, changes in adolescent body composition following this treatment are expected to be larger than those seen in adults. This study has not yet been conducted. If, hypothetically, the adolescents' response to the albuterol/caffeine combination were twice the response seen in the single adult of Example 3, then one would expect to see an increase in lean body tissue of 2.5% after 8 weeks, and approximately the same percentage decrease in body fat mass. These initial changes would project to about a 5% greater reduction in body weight than with placebo after 6 months (with weight loss coming from body fat); then after about 6 months fat loss is expected to plateau. That result would represent a greater reduction than Orlistat gives in adults. (For Orlistat the figures are about 2.8% weight loss after 6 months, 2.1% of which is attributable to fat loss, and 0.7% of which is attributable to reduction in lean tissue.)

### EXAMPLES 8 AND 9

### Children and Adults

Following the successful conclusion of Example 7, preliminary trials in adolescents, generally similar trials will then be conducted both in pre-adolescent children and also in post-adolescent adults. It is expected that the growing groups (adolescents and children) will likely show a greater response in both increased muscle mass and decreased fat mass as compared to adults who have finished growth. Also, adults who typically look at the “bathroom scale” to assess “fat loss” might find a lack of overall weight loss disappointing, despite the loss of fat and the increase in lean tissue. This subjective factor could perhaps be more significant in adults than in children or adolescents.

### EXAMPLE 10

### Sarcopenia

A randomized, double-blind, placebo-controlled study is conducted in 20 individuals with sarcopenia and frailty: age 65-85 years, habitual gait speed less than 1 m/s, appendicular lean mass/height² below 7.23 kg/m² in men, and below 5.67 kg/m² in women. Consent is discussed and obtained at the initial screening visit, and qualification for the study is determined. If the subject meets all of the inclusion criteria and none of the exclusion criteria, he or she will be enrolled in the study. Body composition and functional testing will occur before and then 8 weeks after taking the assigned intervention.

The safety of each medication, the placebo, and the combination of the two medications will be assessed. The hypothesis is that the combination of caffeine and albuterol will cause the greatest increase in lean body mass and increase in functional ability, more than the individual medications alone, and more than the sum of the effects from the individual medications (synergy). Data from this pilot study will serve as preliminary data for a larger future study.

Participants will attend an initial screening visit (SV) after an overnight fast of 10 hours. Consent will be obtained and the inclusion/exclusion criteria will be assessed. Screening labs will help determine eligibility: CBC; Chem 26 including glucose, BUN, creatinine, sodium, chloride, potassium, carbon dioxide, uric acid, total protein, phosphorus, albumin, calcium, magnesium, total bilirubin, CPK, LDH, AST, ALT, alkaline phosphatase, GGT, amylase, iron, cholesterol, triglycerides, HDL, LDL; fasting insulin; DEXA, 4-gait speed test; strength measures including leg press, chest press and hand grip strength; functional measures including a stair climb, 6 minute walk test, short physical performance battery; urinalysis; and an EKG.

### Inclusion Criteria:

Healthy male or female between 65 and 85 years of age inclusive
Habitual gait speed under 1 meter per second
Appendicular lean mass/height² below 7.23 kg/m² in men, and below 5.67 kg/m² in women

### Exclusion Criteria:

Taking a medication known to affect weight or body composition such as systemic glucocorticoids, atypical anti-psychotics, or weight loss medications
Taking beta-stimulators or beta-blockers on a regular basis
Taking any chronic medication that has not had a stable dose for 1 month or longer
Type 1 or type 2 diabetic
Any significant cardiac disease (e.g., heart failure, arrhythmias, or valve disease), uncontrolled pulmonary disease, chronic liver disease, chronic kidney disease, or chronic infectious disease
Any significant psychiatric illness that is unstable or untreated, e.g., bipolar disorder, severe depression, or severe anxiety
Otherwise considered unfit to participate in the study, based on evaluation by the medical investigator

Study Design

Twenty sarcopenic and frail subjects will be recruited through general advertisements and physician referrals. The study will comprise six total study visits. The first study visit will be a screening visit, and will include a medical history/physical examination, an electrocardiogram, DEXA (dual-energy X-ray absorptiometry, used to assay bone mineral density, lean body tissue, and fat body tissue), gait speed, strength measures, functional measures and screening labs. Subjects who qualify will return for a baseline (fasting) visit within one month of the initial screening visit, and will be randomized to one of the four, equally-sized groups:

Group 1: Placebo-placebo three times per day (tid) orally. Group 2: Caffeine 100 mg-placebo tid orally. Group 3: Albuterol 2 mg-placebo tid orally. Group 4: Caffeine 100 mg tid and Albuterol 2 mg tid orally. Albuterol will be increased to 4 mg after 2 weeks, if the subject is tolerating the treatment well. Subjects will be advised to limit their intact of any additional caffeine.

Subjects will be advised to limit their intact of any additional caffeine. It should not be necessary to restrict subjects' intake of additional caffeine for safety concerns. The 300 mg/day used in this combination is substantially less than the amount of non-prescription caffeine one could consume in accordance with package directions (up to 1600 mg/day). Nevertheless, it would be desirable to restrict other sources of caffeine would help to maintain the optimal ratio of albuterol to caffeine.

Starting with the baseline visit and repeating at each 2 week visit, the subject will receive instruction from trained personnel in exercises designed to improve strength and function in those with sarcopenia. At each of weeks 2, 4, 6, and 8, compliance (via pill counts) and adverse events will be assessed. In addition to these assessments, at each of weeks 4 and 8 the subject will meet with the exercise testing personnel regarding their strength and function program. Finally, at the last study visit, after 8 weeks of intervention, the subject will have an electrocardiogram, a repeat DXA, a gait speed test, strength testing, functional testing and repeat labs to compare with the initial screening labs.

Because this will be a pilot study, no power calculation will be done to determine the expected number of participants for statistical significance. Even if there should be no statistically significant difference between groups, the pilot study is nevertheless expected to show proof of concept, and to suggest the variance and differences between the groups needed to power a statistically significant follow-up study. The primary endpoint will be percent change in total lean body mass over the 8-week period via DXA scan using Student t-tests. In addition, secondary endpoints will include QTc interval changes from baseline, appendicular lean mass by DEXA, gait speed, fat mass by DEXA, strength testing, and functional testing in the three treatment groups compared with placebo using Student t-tests. Any adverse events and side effects will be compared using a chi-squared test or other appropriate statistical test. Statistical significance will be set at p<0.05; although statistical significance is not expected in this small-scale pilot study. Demographics will be compared by t-test if normally distributed. Any non-normally distributed data will be analyzed by chi-squared test.

Outcomes

This study has not yet been conducted. The exercise instruction is expected to increase strength and possibly lean mass as well as strength and function. The addition of albuterol is expected to increase these changes, and a further increase is expected with the combination of caffeine and albuterol.

Miscellaneous

Compositions used in the present invention may be administered to a patient by any suitable means, including oral, topical, intravenous, parenteral, dermal, epidermal, and subcutaneous administration. Parenteral infusions include intramuscular, intravenous, intraarterial, or intraperitoneal administration. The two components may be administered separately or together. In a preferred embodiment, the two components are administered together as an admixture, and are administered orally in a pill or capsule formulation.

Pharmaceutically acceptable carrier preparations include sterile, aqueous or non-aqueous solutions, suspensions, emulsions, fillers, and binders. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredients may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti- oxidants, chelating agents, inert gases, and the like.

The form may vary depending upon the route of administration. For example, compositions for injection may be provided in the form of an ampoule, each containing a unit dose amount, or in the form of a container containing multiple doses.

The components of the present invention may be formulated into therapeutic compositions as pharmaceutically acceptable salts. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in nanoparticles or microcapsules by techniques otherwise known in the art including, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The two components of the therapy are caffeine and albuterol (salbutamol). Caffeine does not have optical isomers. Albuterol has two optical isomers. For economic reasons, it is generally preferred to administer albuterol as a racemic mixture of the two optical isomers; however, it is also possible to administer albuterol in other proportions of the two isomers, up to and including 0% R and 100% S, or up to and including 100% R and 0% S. The (R)-enantiomer is believed to be primarily responsible for pharmacologic activity; however the (S)-enantiomer is also believed to play a role, namely by blocking metabolic pathways associated with the elimination of both enantiomers. It is also possible that the (S)-enantiomer may play other roles not yet appreciated. Unless otherwise clearly stated in a particular claim, a reference in a claim to “albuterol” or “salbutamol” refers to any proportions of the two enantiomers—a racemic mixture (50%-50%), 100% (R) enantiomer, 100% (S) enantiomer, or a mixture of any other proportions of the (R) and (S) enantiomers.

As used herein, an “effective amount” of a composition is an amount, that when administered to a patient over a period of time reduces body fat or increases lean body mass to a clinically significant degree; or alternatively, to a statistically significant degree as compared to control. “Statistical significance” means significance at the P<0.05 level, or such other measure of statistical significance as would be used by those of skill in the art of biomedical statistics in the context of the treatment.

Alternatively, a treatment can be considered to produce an “improvement” in a patient's condition if, without necessarily reversing symptoms such as those described in the previous paragraph, the treatment halts or slows the further progression of those symptoms.

The terms “increase,” “increases,” “increased,” “increasing,” and similar terms indicate an elevation of at least 0.5%, 1%, 1.5%, 2%, 2.5%, 3%, 4%, 5%, or more.

The terms “reduce,” “reduces, ” “reduced,” “reduction,” and similar terms mean a decrease of at least 0.5%, 1%, 1.5%, 2%, 2.5%, 3%, 4%, 5%, or more.

“Effective amount” refers to an amount of a composition that is sufficient to produce a desired effect, which can be a therapeutic or beneficial effect. The effective amount will vary with the age, general condition of the subject, the severity of the condition being treated, the duration of the treatment, the nature of any concurrent treatment, the pharmaceutically acceptable carrier used, and like factors within the knowledge and expertise of those skilled in the art. As appropriate, an “effective amount” in any individual case can be determined by one of skill in the art by reference to the pertinent texts and literature or by using routine experimentation.

By the term “treat,” “treating,” or “treatment of (and grammatical variations thereof) it is meant that the severity of the subject's condition is reduced, at least partially improved or ameliorated, or that some alleviation, mitigation or decrease in at least one clinical symptom is achieved or there is a delay in the progression of the disease or disorder.

A “therapeutically effective” amount as used herein is an amount that is sufficient to treat (as defined herein) the subject. Those skilled in the art will appreciate that the therapeutic effects need not be complete or curative, as long as some benefit is provided to the subject.

“Body mass index” or “BMI” means the ratio of a person's mass in kilograms divided by the square of the person's height in meters.

The complete disclosures of all references cited in this specification are hereby incorporated by reference. Also incorporated by reference are the complete disclosures of the priority applications, U.S. provisional patent application Ser. No. 62/147,151, filed 14 Apr. 2015; and U.S. provisional patent application Ser. No. 62/270,897, filed 22 Dec. 2015. Also incorporated by reference is the complete disclosure of the following publication by the inventor and colleagues: A. Liu et al., “The effect of caffeine and albuterol on body composition and metabolic rate,” Obesity, vol. 23(9), pp. 1830-1835 (2015). See also a May 2014 clinical trial notice by F. Greenway, “Testing Potential Synergistic Effects of Albuterol and Caffeine on Metabolic Rate,” https://clinicaltrials.gov/ct2/show/NCT02135965. In the event of an otherwise irreconcilable conflict, however, the present specification shall control.

What is claimed:

1. A composition comprising albuterol and caffeine; wherein the mass ratio of albuterol to caffeine is between 1:20 and 1:30.

2. The composition of claim 1, wherein the mass ratio of albuterol to caffeine is 1:25.

3. The composition of claim 1, wherein said albuterol consists of 4 mg albuterol.

4. The composition of claim 1, wherein said caffeine consists of 100 mg caffeine.

5. The composition of claim 1, wherein said composition is a timed-release formulation adapted to cause the release of said albuterol, said caffeine, or both over a period of time in the digestive tract.

6. The composition of claim 5, wherein said albuterol consists of 12 mg albuterol, and wherein said caffeine consists of 300 mg caffeine.

7. The composition of claim 1, wherein said composition is suitable for oral administration.

8. The composition of claim 1, wherein said composition contains no additional pharmacologically active components, other than said albuterol and said caffeine.

9. A method for improving the body composition of a mammalian patient, comprising administering to the patient over a period of time albuterol, and administering to the patient over a period of time caffeine; wherein the mass ratio of albuterol to caffeine is between 1:20 and 1:30; and wherein the body composition of the human is improved in one or both of the following ways over a period of time: reduction in body fat, or increase in lean body mass.

10. The method of claim 9, wherein the mass ratio of albuterol to caffeine is 1:25.

11. The method of claim 9, wherein each albuterol-administering step consists of administering 4 mg albuterol.

12. The method of claim 9, wherein each caffeine-administering step consists of administering 100 mg caffeine.

13. The method of claim 9, wherein said administering step occurs three times daily.

14. The method of claim 9, wherein said administering comprises administering a timed-release formulation containing both the albuterol and the caffeine.

15. The method of claim 14, wherein the albuterol in the timed-release formulation consists of 12 mg albuterol, and wherein the caffeine in the timed- release formulation consists of 300 mg caffeine, and wherein said administering step occurs once daily.

16. The method of claim 9, wherein the caffeine and albuterol are administered orally.

17. The method of claim 9, wherein the caffeine and albuterol are administered orally as an admixture in a tablet or capsule.

18. The method of claim 9, wherein the caffeine and albuterol are administered orally as a timed-release admixture in a tablet or capsule.

19. The method of claim 18, wherein the tablet or capsule contains no additional pharmacologically active components, other than the albuterol and the caffeine

20. The method of claim 9, wherein the patient is an obese adolescent, and wherein, following use of the method over a period of time, the symptoms of obesity improve.

21. The method of claim 9, wherein the patient is an obese pre-adolescent juvenile, and wherein, following use of the method over a period of time, the symptoms of obesity improve.

22. The method of claim 9, wherein the patient is an adult, and wherein, following use of the method over a period of time, the symptoms of obesity improve.

23. The method of claim 9, wherein the patient has sarcopenia, and wherein, following use of the method over a period of time, the symptoms of sarcopenia improve.

24. The method of claim 9, wherein the patient is a human.

25. The method of claim 9, wherein the patient is a cat or a dog.
